(19) United States
(12) Patent Application Publication
(10) Pub. No.: US 2022/0184166 A1
(43) Pub. Date: Jun. 16, 2022

(54) DISCRETE PHASE PARTICLES INCLUDING COMPOUNDS FROM OLEA EUROPAEA

(71) Applicant: Theo Rallis, Windsor (CA)

(72) Inventors: Theo Rallis, Windsor (CA); Joshua James, San Antonio, TX (US)

(21) Appl. No.: 17/547,722

(22) Filed: Dec. 10, 2021

Related U.S. Application Data
(60) Provisional application No. 63/123,798, filed on Dec. 10, 2020.

(51) Int. Cl. A61K 36/63; A61K 9/107

(52) U.S. Cl. CPC A61K 36/63; A61K 9/107

(57) ABSTRACT

Compositions, methods of making, and uses are provided that relate to a continuous medium forming a first phase and a discrete phase particle containing a second phase. The discrete phase particle includes one or more compounds derived from Olea europaea, such as oleocanthal, oleacein, certain amounts of oleocanthal and oleacein, and polyphenols. Such compositions can be used to administer an active ingredient to a subject, including topical and oral administration routes. The discrete phase compound including the one or more compounds derived from Olea europaea can exhibit certain antioxidant and/or anti-inflammatory effects in maximizing delivery and uptake of the composition, where such effects can protect the active ingredient contained therein as well as provide advantageous secondary or compound effects in addition to those provided by the active ingredient.

### CROSS-REFERENCE TO RELATED APPLICATIONS

This application claims the benefit of U.S. Provisional Application No. 63/123,798, filed on Dec. 10, 2020. The entire disclosure of the above application is incorporated herein by reference.

### FIELD

The present technology relates to discrete phase particles, in particular where the discrete phase particles are in the form of micelles, reverse micelles, liposomes, and/or reverse liposomes, and further include compounds derived from Olea europaea.

### INTRODUCTION

This section provides background information related to the present disclosure, which is not necessarily prior art.

Various amphiphilic molecules can be assembled and/or self-assemble to form discrete phase particles within a continuous medium, where such particles include micelles and liposomes. A micelle includes a collective structure of surfactant molecules dispersed in a liquid that can form a colloidal suspension. Surfactant molecules, for example, can be amphiphilic and can contain one or more hydrophilic portions and one or more hydrophobic portions. A typical micelle in an aqueous continuous phase can form an aggregate or collective structure where hydrophilic “head” regions are in contact with a surrounding aqueous solvent and hydrophobic “tail” regions are sequestered in a center of the structure. The micelle structure can result from the packing behavior of amphiphilic molecules (e.g., phospholipids) into layers or into a bilayer. Intermolecular interactions and packing considerations between the volume of an interior of the bilayer, including the “tail” regions in conjunction with accommodating an area of the “head” regions by the hydration of a lipid head group, can lead to the formation of the micelle. This type of micelle can be referred to as a normal-phase micelle, e.g., oil-in-water micelle, where the micelle sequesters a hydrophobic center dispersed within a continuous aqueous medium. However, it is possible to form reverse micelles that include the hydrophilic “head” groups at the particle center with the hydrophilic “tails” extending out, e.g., water-in-oil micelle, where the micelle sequesters a hydrophilic center dispersed within a continuous nonpolar or nonaqueous medium.

Micelles can be approximately spherical in shape, although other forms such as ellipsoids, cylinders, and bilayers, are also possible. Shape and size of a micelle can be a function of the molecular geometry of different surfactant molecules, including mixtures of different types of phospholipids used to form the micelle, for example, as well as solution conditions such as surfactant concentration, temperature, pH, and ionic strength. The process of forming micelles can be referred to as micellization and is part of the phase behavior of lipids and polymorphism thereof.

A liposome is a particular collective structure of surfactant molecules, similar in part to a micelle, but where at least one lipid bilayer encapsulates an interior volume. The liposome can be formed where the interior volume can be the same phase as the exterior phase of the liposome. In this way, for example, the liposome can encapsulate or contain molecules soluble in an aqueous medium, but where the molecules solubilized within the interior volume are protected or inaccessible from the exterior of the liposome. Liposomes can therefore be used as a vehicle for administration of certain water-soluble compounds; e.g., nutrients, pharmaceuticals, and other functional molecules.

There are various ways to form liposomes. Liposomes can be prepared by disrupting biological membranes (e.g., by sonication) and/or by aggregating certain surfactants from various sources in various ways. Various amphiphilic molecules, such as phospholipids (e.g., phosphatidylcholine), can be used to form liposomes; however, other lipids, molecules having hydrophobic portions, and hydrophobic molecules can be included that are compatible with the lipid bilayer structure. Liposomes can also include one or more surface modifications or ligands that facilitate binding, uptake, and/or fusion with other structures or biological processes. Types of liposomes include unilamellar liposome vesicles with one lipid bilayer and multilamellar vesicles having more than one lamellar phase lipid bilayer.

Various types of hydrophilic, hydrophobic, and amphiphilic molecules can be used to form and/or included with discrete phase particles dispersed within a continuous medium. For example, micelles and liposomes can include one or more lipids, phospholipids, proteins having hydrophobic portions and/or covalently attached hydrophobic groups (e.g., myristoylation), hydrophobic molecules (e.g., phenolic compounds), fat soluble vitamins, etc. present within the lipid portion of the micelle or liposome, whereas various hydrophilic and/or water soluble molecules can be encapsulated by the liposome bilayer or associated with the hydrophilic portion (e.g., “head” region) of the micelle or liposome.

Olives, the fruit of the Olea europaea tree, include various lipids, fatty acids, and phenolic compounds, among other hydrophobic and amphiphilic molecules. Olive oil can include various phospholipids, such as phosphatidic acid, lyso-phosphatidic acid, and phosphatidylinositol, and can include a mixture of triglyceride esters of oleic acid, linoleic acid, palmitic acid, as well as other fatty acids, squalene, and sterols (e.g., phytosterol, tocosterols). The composition of olive oil can vary by cultivar, region, altitude, time of harvest, and extraction process. Certain olive oil components, including profiles or ratios of certain components with respect to each other, can result from particular types of olives as well as particular types of extraction processes. Olive oil can include various antioxidant compounds along with a certain amount of phenolics (e.g., about 0.5%).

Among the antioxidant compounds that have been isolated from olive oil are various phenolic compounds, including Vitamin E (alpha-tocopherol), carotenoids, and phenolic compounds (simple phenols such as hydroxytyrosol and complex phenols such as oleuropein). The phenolic content of olive oils varies according to the climatic conditions in the producing area, when the olives are harvested, and how ripe the olives are when picked. Particular olive oil extraction processes and storage methods can also have an influence. Phenolic compounds have been linked with many biological properties; for instance, hydroxytyrosol inhibits platelet aggregation and has anti-inflammatory properties, where oleuropein encourages the formation of nitric acid, which is a powerful vasodilator and exerts a strong antibacterial effect.

It would be desirable to have ways of preparing discrete phase particles, in particular micelles, liposomes, and variants thereof, where such discrete phase particles are formed using compounds, such as various lipids, derived from olive oil, where a portion of each particle, and/or where a portion of the particles is derived from olive oil, in order to optimize the certain antioxidant properties of olive oil in conjunction with micelles and/or liposomes, including such micelles and/or liposomes loaded with active ingredients such as one or more nutrients, pharmaceuticals, and/or other functional molecules.

### SUMMARY

The present technology provides ways of making and using discrete phase particles in various forms, including micelles, reverse micelles, liposomes, and/or reverse liposomes, which further include one or more compounds derived from Olea europaea.

Compositions are provided that include a continuous medium forming a first phase and a discrete phase particle containing a second phase, where the discrete phase particle includes a compound derived from Olea europaea. The discrete phase particles in relation to the first phase and the second phase can be configured in various ways. The first phase can include an aqueous phase, the second phase can include a nonaqueous phase, and the discrete phase particle can be in the form of a micelle. The first phase can include a nonaqueous phase, the second phase can include an aqueous phase, and the discrete phase particle can be in the form of a reverse micelle. The first phase can be an aqueous phase, the second phase can be a nonaqueous phase, the discrete phase particle can be in the form of a liposome, and the liposome can include a third phase that includes an aqueous phase. And the first phase can be a nonaqueous phase, the second phase can be an aqueous phase, the discrete phase particle can be in the form of a reverse liposome, and the reverse liposome can include a third phase that includes a nonaqueous phase. The various discrete phase particles can include one or more active ingredients, where each active ingredient can be associated with a certain phase dependent on the relative hydrophilicity/hydrophobicity thereof.

Methods of administering an active ingredient to a subject are provided that include providing one or more compositions including one or more discrete phase particles, as set forth herein. The one or more discrete phase particles can include one or more active ingredients. The one or more compositions including one or more discrete phase particles can be administered to the subject, thereby administering the one or more active ingredients to the subject. Various types of administration can be used, including topical and oral, further including where oral administration employs a composition encapsulated with an enteric coating.

Compositions are provided that include a plurality of discrete phase particles including at least one compound derived from Olea europaea. The plurality of discrete phase particles can include one or more of micelles, reverse micelles, liposomes, and reverse liposomes. The at least one compound derived from Olea europaea can include one or more of a phospholipid, a phenolic compound, and a fatty acid. In certain embodiments, the at least one compound derived from Olea europaea includes olive oil. The olive oil can include ice-pressed olive oil, such as ice-pressed olive oil derived from Greek olives. The ice-pressed olive oil derived from Greek olives can have a total amount of polyphenols (index D3) greater than about 500 mg/kg, including where the total amount of polyphenols (index D3) can be from about 500-2,000 mg/kg. Certain embodiments include where the ice-pressed olive oil derived from Greek olives comprises: oleocanthal from about 130-245 mg/kg; oleacein from about 90-165 mg/kg; oleocanthal+oleacein (index D1) from about 220-410 mg/kg; and total polyphenols analyzed (index D3) from about 760-1420 mg/kg. The composition can be encapsulated and can be administered to an individual. Certain embodiments include where the plurality of discrete phase particles further comprises an active ingredient such as a nutrient, vitamin, pharmaceutical, and/or other active ingredients, where such particles can be administered to deliver the one or more active ingredients to an individual for treatment or prophylaxis of a particular condition. Administration can be oral, where for example, the composition can be encapsulated for oral administration via emulsion, capsule, tablet, gel cap, etc. Accordingly, the at least one compound derived from Olea europaea can provide certain benefits in conjunction with the discrete phase particles in delivering the vitamin, pharmaceutical, and/or the active ingredient to an individual, including optimizing passage through the individual's stomach, uptake in the intestine, and antioxidant and/or anti-inflammatory effects in maximizing delivery and uptake of the composition.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A, 1B, and 1C include schematic perspective cross-sections of a liposome (FIG. 1A), a micelle (FIG. 1B), and a bilayer sheet (FIG. 1C).

FIGS. 2A, 2B, and 2C include schematic cross-sections of a reverse micelle in aprotic continuous phase (FIG. 2A), a normal micelle in an aqueous continuous phase (FIG. 2B), and a liposome in an aqueous phase (FIG. 2C).

FIG. 3 provides a schematic depiction of a reverse liposome showing a reverse phospholipid bilayer with an active ingredient enclosed by the reverse phospholipid bilayer.

FIGS. 4A and 4B include schematic cross-sections of a liposome including a bilayer enclosing an active ingredient (FIG. 4A) and multilamellar discrete phase particle including multiple enclosures of an active ingredient (FIG. 4B).

FIG. 5 is a schematic of a portion of a reverse bilayer, where saponin molecules (represented by a single tail) are included with phospholipids (represented by double tails) in the reverse bilayer, cations (e.g., magnesium ions) provide bridging interactions between the polar or hydrophilic head portions of the reverse bilayer, and PEGylated phospholipids are included in the reverse bilayer.

FIG. 6 is a schematic of a reverse liposome showing a reverse phospholipid bilayer with an active ingredient enclosed by the reverse bilayer, where PEGylated phospholipids are present in the reverse bilayer and saponin molecules are associated with the reverse bilayer.

FIG. 7 is a schematic of a liposome showing a phospholipid bilayer with hydrophilic molecules enclosed by the bilayer and hydrophobic molecules associated with the lipid portion of the bilayer.

FIG. 8 is a graphical representation of the uptake of normal vitamin C versus liposomal vitamin C.

FIG. 9 is a graphical representation of oxidative stress following the uptake of liposomal vitamin C versus a placebo.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on. As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

The present technology relates to compositions and ways of making and using such compositions that include one or more discrete phase particles, in particular where the discrete phase particles are in the form of micelles, reverse micelles, liposomes, and/or reverse liposomes, and further include one or more compounds derived from Olea europaea. Compositions are provided that include a continuous medium forming a first phase and a discrete phase particle containing a second phase, where the discrete phase particle includes a compound derived from Olea europaea. Various phases and discrete phase particles can be formed. A first example includes the first phase is an aqueous phase, the second phase is a nonaqueous phase, and the discrete phase particle is in the form of a micelle. A second example includes where the first phase is a nonaqueous phase, the second phase is an aqueous phase, and the discrete phase particle is in the form of a reverse micelle. A third example includes where the first phase is an aqueous phase, the second phase is a nonaqueous phase, the discrete phase particle is in the form of a liposome, and the liposome includes a third phase that is an aqueous phase. A fourth example includes where the first phase is a nonaqueous phase, the second phase is an aqueous phase, the discrete phase particle is in the form of a reverse liposome, and the reverse liposome includes a third phase that is a nonaqueous phase. Additional examples include various mixtures of the aforementioned first and third examples and various mixtures of the second and fourth examples.

The compound derived from Olea europaea can include various aspects. Certain embodiments include where the compound derived from Olea europaea includes a phospholipid, a phenolic compound, a fatty acid, and/or a terpenoid/isoprenoid compound. The compound derived from Olea europaea can include olive oil, including unfractionated olive oil or fractionated olive oil. Fractionated olive oil can include a portion of olive oil that is missing a component found in unfractionated olive oil or where the fractionated olive oil includes a different ratio of compounds relative to unfractionated olive oil. The olive oil can include olive oil prepared by ice-pressing; e.g., where the olive oil is extracted from the olive fruit at a reduced temperature compared to an ambient temperature, in particular where the olive oil is extracted from the olive fruit at a temperature of about 0° C. Certain embodiments include where the ice-pressed olive oil is derived from Greek olives. For example, ice-pressed olive oil derived from Greek olives demonstrates particular component profiles versus olive oil prepared in other ways and sourced from other locales. Ice-pressed olive oil derived from Greek olives can specifically include a total amount of polyphenols (index D3) greater than about 500 mg/kg, as well as a total amount of polyphenols (index D3) from about 500-2,000 mg/kg, in certain embodiments. The ice-pressed olive oil derived from Greek olives can include oleocanthal from about 130 mg/kg to about 245 mg/kg, oleacein from about 90 mg/kg to about 165 mg/kg, oleocanthal+oleacein (index D1) from about 220 mg/kg to about 410 mg/kg, and total polyphenols analyzed (index D3) from about 760 mg/kg to about 1420 mg/kg. These particular component profiles can provide unique antioxidant properties as well as unique anti-inflammatory properties, which directly result from olive oil prepared and sourced according to the present technology.

The discrete phase particle can include various aspects, including the addition of other compounds and components not derived from Olea europaea. For example, micelles, reverse micelles, liposomes, and/or reverse liposomes can be formed with phospholipids from natural sources other than Olea europaea, as well as formed using modified versions of natural compounds and components, and further including various synthetic compounds and components. Certain embodiments include where the discrete phase particle includes a PEGylated phospholipid; e.g., the covalent and/or non-covalent attachment or amalgamation of polyethylene glycol (PEG) polymer chains to a phospholipid. The discrete phase particle can also include one or more saponins. Saponins include triterpene glycosides, which have amphipathic properties and can function as surfactants with the ability to interact with cell membrane components, such as cholesterol and phospholipids, including portions of the discrete phase particles provided herein. The discrete phase particle can include one or more active ingredients, such as various nutrients, vitamins, minerals, nutraceuticals, pharmaceuticals, as well as other functional molecules. In certain embodiments, the active ingredient is present in the second phase, such that the active ingredient is contained by the discrete phase particle. Other embodiments include where one or more certain active ingredients can be associated with a hydrophilic portion or phase of the discrete phase particle while one or more other active ingredients can be associated with a hydrophobic portion or phase of the discrete phase particle.

In certain embodiments, compositions of the present technology can be encapsulated in various ways, including where such compositions are administered to a subject. For example, various geltab or capsule forms, sizes, and constructions can be used to provide unit dose forms of the compositions, where the continuous medium forming the first phase and the discrete phase particle(s) containing the second phase are encapsulated. This can be accomplished using gelatin or vegan capsules, including one- and two-piece gel capsules. Other components can be encapsulated along with the composition, including one or more excipients, stabilizers, pH buffering agents, etc. Embodiments further include where the composition is encapsulated by an enteric coating. The enteric coating can allow the composition to pass through the stomach before being released in the lower gastrointestinal tract of the subject.

Methods of administering an active ingredient to a subject are provided that can include providing a composition including a continuous medium forming a first phase and a discrete phase particle containing a second phase, where the discrete phase particle includes a compound derived from Olea europaea. The various compositions provided herein can be used in administering the composition to the subject, thereby administering the active ingredient to the subject. Multiple modalities of administration can be employed, including where the administering is topical, where the administering is oral, and where the administering is oral and the composition is encapsulated with an enteric coating. Where the composition is applied topically, the composition can be applied directly or can be formulated along with various emollients prior to application.

Aspects of the present technology further provide compositions, ways of making compositions, and methods of using compositions that include a plurality of discrete phase particles including at least one compound derived from Olea europaea. The plurality of discrete phase particles can include one or more micelles, reverse micelles, liposomes, and/or reverse liposomes. The present compositions can include various nutrients, compounds, and phospholipids and can be used to deliver such molecules to an individual by oral ingestion, for example. With respect to delivery of compounds (e.g., active ingredients), including various nutrients (e.g., vitamins, minerals, etc.), using the present discrete phase particles, one or more compounds can be associated with or enclosed in the discrete phase particles. Depending on the nature of the compound, for example, the compound can be enclosed within the discrete phase particle (e.g., enclosed by a liposomal bilayer) or embedded within a part of the discrete phase particle (e.g., associated with a lipid portion of a liposomal bilayer). In this way, discrete phase particles including one or more active ingredients can protect the one or more active ingredients from the gut and uptake thereof can be improved. With respect to delivery of certain compounds using discrete phase particles, such compounds can be stabilized by the particles and can be protected during passage through a portion of the gastrointestinal tract following ingestion, for example. Such compounds can be enclosed by or associated with different portions of the particles. With respect to phospholipids, fatty acid chains and molecules like phosphatidylcholine can form at least part of the particles. Such phospholipids and fatty acids can also provide a nutritive function, including provision of antioxidant and/or anti-inflammatory activities in addition to functioning as a delivery vehicle.

In certain embodiments, the plurality of discrete phase particles can include a micelle. The micelle can include an aggregate of surfactant molecules dispersed in a liquid, forming a colloidal suspension. The plurality of discrete phase particles can also include a reverse micelle. Reverse micelles can be used for selective separation and purification of biomolecules, and for the synthesis of nanoparticles. Reverse micelles can include nanometer-size droplets of aqueous phase, stabilized by surfactants in a nonaqueous phase. Micelles can be used as carriers for antioxidants and other molecules. Micelles can have a more rapid delivery or circulation time inside an individual versus liposomes, owing in part to their smaller size over liposomes. Moreover, micelles can contain a hydrophobic core stabilized through hydrophobic units.

The present compositions including discrete phase particles can include reverse micellular or liposomal lipid nano-encapsulation technology, and can include phospholipids (e.g., phosphatidylcholine) from Olea europaea that can be used alone or in conjunction with phospholipids from other sources (e.g., sunflower) and can be prepared by various physiochemical processes to form multi-phase discrete particles. Aspects of the present compositions include the combination of a micelle and reverse micelle to create a lipid bilayer liposome. The discrete phase particles can be used to encapsulate both active and non-active ingredients, where the discrete phase particles can provide a more passive uptake or immersion into the body by moving a fat thru a fat versus forcing an aqueous molecule though a fat cell.

Advantages of lipid nano-encapsulation according to the present technology can further include a reduction of oxidation and degradation of encapsulated ingredients, improving shelf life, an increase in the palatability of molecules/ingredients having typically aversive tastes and aromas, an increase in volume of distribution, an increase in duration of action, a reduction in required dosing and load of an active ingredient, a reduction in initial spike curve of absorption (e.g., fast acting and long lasting), and/or the ability to solubilize more hydrophobic molecules/ingredients. Certain active molecules/ingredients can exhibit limited solubilities, where enclosure by or association thereof with discrete phase particles can enhancing solubility and hence bioavailability of such molecules/ingredients. In particular, reverse micelles can be used for the encapsulation and delivery of hydrophilic active ingredients.

Phospholipids used in the present technology can provide certain aspects to the discrete phase particles, including certain antioxidative effects. A mechanism of antioxidative effects of phospholipids not fully understood, but the polar group of phospholipids can play an important role and nitrogen-containing phospholipids such as phosphatidylcholine and phosphatidylethanolamine can function as efficient antioxidants under most conditions. Phospholipids can also decrease oil oxidation by sequestering metals, where a concentration for the maximal antioxidant activity can be between 3 and 60 ppm. For example, certain oxidation of certain oils can be decreased with addition of 5 to 10 ppm phospholipids, and higher amounts of phospholipids can act as prooxidants. As described herein, phospholipids have hydrophilic and hydrophobic groups in the same molecule, where the hydrophilic groups of the phospholipids can be on the surface of the discrete phase particles and hydrophobic groups can be in an oil portion. Phospholipids can further decrease a surface tension of lipids or oils.

The discrete phase particles can include a reverse liposome having a reverse phospholipid bilayer with an active pharmaceutical ingredient enclosed by the reverse bilayer, where PEGylated phospholipids are present in the reverse bilayer and saponin molecules are associated with the reverse bilayer. The saponin can allow nano encapsulations to spontaneously form with high shear. Iced sonication (e.g., chilling a vacuum homogenizer) can also be used. PEGylated phospholipids (i.e., polyethylene glycol (PEG) polymer covalently attached to the head-group of a phospholipid) can be incorporated. PEGylated phospholipids can offer what is known as a stealth effect to discrete phase particles having molecules/ingredients enclosed therein or associated therewith as the discrete phase particles circulate within the body. The human immune system is driven to protect the body from any foreign object, and medicinal nanoparticles are no exception. To aid in delivery efficiency and to allow more circulation time for cargo molecules to reach intended diseases sites, PEG can be incorporated to shield these nanoparticles by preventing blood plasma proteins from absorbing into the liposome surface, increasing bloodstream circulation lifetime. Another benefit of PEGylation includes a boost in stability for liposome-like nanostructures. Conventional liposomes, particularly those smaller than 200 nm in size, can be unstable on their own and tend to fuse with each other to reduce surface tension. Fusion can be minimized by incorporation of PEGylated phospholipids.

In certain embodiments, the plurality of discrete phase particles including at least one compound derived from Olea europaea can exhibit a unique antioxidant profile based upon the source and preparation method of oil derived from Olea europaea. This is especially true compared to olive oils prepared from various sources and in various ways. Olive oil components can provide antioxidant properties that can operate in conjunction with active molecules or ingredients. Such combinations can be administered for various complementary and synergistic antioxidant effects, including the treatment, management, and/or prophylaxis of various conditions.

Oil made from the fruit of the tree Olea europaea, referred to as olive oil, can provide a source of liposoluble antioxidants and beneficial lipophilic compounds. Olive oil can further solubilize desirable hydrophobic compounds, including various hydrophobic antioxidants and vitamins. Components of olive oil that can provide beneficial aspects include monounsaturated fats (e.g., oleic acid), vitamins E and K, anti-inflammatory compounds (e.g., oleocanthal), compounds related to preventing low density lipoprotein (LDL) cholesterol from oxidizing, compounds related to antibacterial properties, and various phenolic compounds. Types of olive oil phenolic compounds include flavonoids, lignans, simple phenols, and secoiridoids. Phospholipids found in olive oil include phosphatidic acid, lyso-phosphatidic acid, and phosphatidylinositol.

Examples of individual phenolic compounds normally present in olive oil include the following: (i) benzoic acids and derivatives, such as 3-hydroxybenzoic acid, p-hydroxybenzoic acid, 3,4-dihydroxybenzoic acid, gentisic acid, vanillic acid, gallic acid, and syringic acid; (ii) cinnamic acids and derivatives, such as o-coumaric acid, p-coumaric acid, caffeic acid, ferulic acid; and sinapic acid; (iii) phenyl ethyl alcohols, such as tyrosol [(p-hydroxyphenyl)ethanol], and hydroxytyrosol ((3,4-dihydroxyphenyl)ethanol); (iv) other phenol acids and derivatives, such as p-hydroxyphenylacetic acid, 3,4-dihydroxyphenylacetic acid, 4-hydroxy-3-methoxyphenylacetic acid, and 3-(3,4-dihydroxyphenyl)propanoic acid; (v) dialdehydic forms of secoiridoids, such as decarboxymethyloleuropein aglycon (oleacin), and decarboxymethyl ligstroside aglycon (oleocanthal); (vi) secoiridoid aglycons, such as oleuropein aglycon, ligstroside aglycon, aldehydic form of oleuropein aglycon, and aldehydic form of ligstroside aglycon; (vii) flavonoids, such as (+)-taxifolin, apigenin, and luteolin; (viii) lignans, such as (+)-pinoresinol, (+)-1-acetoxypinoresinol, and (+)-1-hydroxypinoresinol; (ix) other categories, such as the hydroxyisochromans 1-phenyl-6,7-dihydroxyisochroman, and 1-(3-methoxy-4-hydroxy)phenyl-6,7-dihydroxy-isochroman may also be present. It should be noted that the total polyphenols of the olive oil of the present technology can include one or more of these phenolic compounds, where the olive oil can be characterized by amounts of oleocanthal, oleacein, oleocanthal+oleacein (index D1), and total polyphenols (index D3), but one or more of these additional compounds can be included in the total polyphenols (index D3) even though amounts of the individual compounds are not assayed or reported. The body of information regarding benefits and concerted action of these phenolic compounds continues to increase, and while not to be limited by theory, it is understood that combinations of polyphenols including more than oleocanthal and/or oleacein may be responsible for the benefits and advantages attributable to the present technology.

Olive oil extraction is the process of extracting the oil present in the olive fruit or olive drupes, known as olive oil. Olive oil is produced in mesocarp cells of the fruit and stored in a particular type of vacuole called a lipovacuole; e.g., every cell contains a tiny olive oil droplet. Olive oil extraction is the process of separating the oil from the other fruit contents; e.g., vegetative aqueous liquid, solid materials, etc. It is possible to attain separation by physical means alone, where oil and water do not typically mix, so they can be relatively easy to separate. This contrasts with other oils that are extracted with chemical solvents; e.g., hexane.

The following general method can be applied to extract olive oil, where the present technology applies an ice-pressing operation to maximize antioxidant activity and extraction. First, olives can be pitted and the fruit processed into a paste; for example, where the olives are ground into an olive paste using various types of millstones at an oil mill. The olive paste can be ground for 30-40 minutes. The grinding can have three objectives: (1) ensure that olives are well ground; (2) allow enough time for oil droplets to join to form larger drops of oil; and (3) allow the olive fruit enzymes to react with precursors to form certain compounds, which can coincide with production of some of the unique aromas and taste of the oil. Olive oil mills can also employ modern crushing methods along with a traditional olive press. After grinding, the olive paste can be spread on fiber disks, which can be stacked on top of each other, then placed into a press. The disks can be made of natural hemp or coconut fiber but can also be made of synthetic fibers that can be easier to clean and maintain. These disks are then put on a hydraulic piston, forming a pile. Pressure is applied on the disks, thus compacting the solid phase of the olive paste and percolating the liquid phases; e.g., oil and vegetation water. The applied hydraulic pressure can be tailored for particular extraction parameters, where in certain instances pressure can be applied up to 400 atm or more. To facilitate separation of the liquid phases, water can be run down the sides of the disks to increase the speed of percolation. Aqueous and nonaqueous portions of the pressed liquid can then be separated either by a decantation process or by means of a centrifuge. One or more pressing steps of the present technology can employ what is referred to herein as ice-pressing.

Ice-pressing can include mixing the olive paste with ice to cool the olive paste, where the olive paste can be cooled down to near 0° C. in certain embodiments. The iced or cooled olive paste can then be spread upon the fiber disks and pressed. The lower temperature of the olive paste during the pressing can slow down and/or inhibit the activity of certain olive fruit enzymes that act upon certain components of the olive paste, including the oil droplets. Likewise, it is possible reduce the temperature of the olives in the initial grinding to form the olive paste to further reduce and/or inhibit the activity certain olive fruit enzymes. Addition of ice to the pressing of the olive paste can further serve to facilitate separation of the aqueous and non-aqueous phases in the pressing, as the ice can form cold water during the pressing that can run down through and out the sides of the disks to increase percolation. The profiles or ratios of certain oxidized and unoxidized components with respect to each other in the olive oil can be substantially changed by the ice pressing operation. Likewise, particular types of olives as well as particular types of extraction parameters can alter the composition of the olive oil.

In certain embodiments, ice-pressed olive oil is prepared from olives sourced from Greece. Such olives prepared in this way can provide a unique profile of antioxidants and other beneficial compounds in comparison to olive oil prepared in other ways and from olives grown in other regions and climates. Table 1 shown below lists an example analysis of ice-pressed olive oil from Greece. The chemical analysis was performed according to the method published in J. Agric. Food Chem., 2012, 60 (47), pp 11696-11703, J. Agric. Food Chem., 2014, 62 (3), pp 600-607, and OLIVAE, 2015, 122, pp 22-33.

TABLE 1
Chemical Analysis of Ice-Pressed Greek Olive Oil

| compound | mg/kg |
|---|---|
| oleocanthal | 188 |
| oleacein | 127 |
| oleocanthal + oleacein (index D1) | 315 |
| ligstroside aglycon (monoaldehyde form) | 62 |
| ligstroside aglycon (dialdehyde form) | 412 |
| oleuropein aglycon (monoaldehyde form) | 102 |
| oleuropein aglycon (dialdehyde form) | 199 |
| total tyrosol derivatives | 662 |
| total hydroxytyrosol derivatives | 428 |
| total polyphenols analyzed (index D3) | 1090 |

In other embodiments, the ice-pressed Greek olive oil can exhibit the values shown in Table 1, where each of the values can vary from between about 1% up to about +/−30%. Other embodiments include where the values vary by +/−30%, +/−20%, +/−10%, and +/−5%. Still further embodiments include where the ice-pressed Greek olive oil exhibits such values for at least two of the compounds in Table 1, including at least three of the compounds, at least four of the compounds, and so on up to where the ice-pressed Greek olive oil exhibits such values for all ten of the compounds or compound types in Table 1.

The olive oil used in the present technology can have a total amount of polyphenols (index D3) greater than about 500 mg/kg. Various embodiments include where the olive oil has a total amount of polyphenols (index D3) from about 500-2,000 mg/kg, from about 750-1,500 mg/kg, and from about 900-1,200 mg/kg. The olive oil can also have a total amount of polyphenols (index D3) of about 1,100 mg/kg. The olive oil can further include one or more of: oleocanthal from about 130-245 mg/kg, oleacein from about 90-165 mg/kg, oleocanthal+oleacein (index D1) from about 220-410 mg/kg, and total polyphenols analyzed (index D3) from about 760-1420 mg/kg. The aforementioned ranges of oleocanthal, oleacein, oleocanthal+oleacein (index D1), and total polyphenols analyzed (index D3) can vary by +/−30%, +/−20%, +/−10%, and +/−5%.

The levels of oleocanthal and oleacein in the ice-pressed Greek olive oil are higher than the levels of these compounds in olive oils produced in other ways and/or produced from olive oils harvested from olives derived from other locations besides Greece. It should be noted that oleocanthal and oleacein exhibit important biological activity and are correlated with anti-inflammatory, antioxidant, cardioprotective, and neuroprotective activities.

Various blends can be made using the olive oil and one or more other oils to include various percentages of each. For example, embodiments include from 1-99% olive oil and 99-1% other oil(s). Other embodiments include about equal parts olive oil and other oil(s). Examples of other oils include various carrier oils and oil blends, such as various non-olive vegetable oils, as well as various lipophilic compounds and hydrophobic compounds. Compositions of the olive oil and the can be administered in various ways, including various oral and topical methods.

Compositions comprising a plurality of discrete phase particles including at least one compound derived from Olea europaea can be prepared in various ways. For example, the discrete phase particles can be formed entirely from compounds derived from olive oil or the discrete phase particles can be formed using one or more compounds derived from olive oil in conjunction with other phospholipids, fatty acids, lipids, and amphiphilic molecules from other sources. These various compounds can be mixed with the olive oil in various ways, where the oils are blended or where the oils are separated from each other by various means. Various emulsions and encapsulations can be formed. In certain embodiments, olive oil compounds and/or other compounds can be emulsified and used to encapsulate various molecules or ingredients using various methods. The discrete phase particles can improve the efficacy and bioavailability of molecules or ingredients enclosed therein or associated therewith when consumed. The discrete phase particles can also increase the half-life and persistence of certain components or ingredients, which can be due in part to certain antioxidant activities of the one or more compounds derived from Olea europaea.

As described herein, liposomes as part of the discrete phase particles can include various active ingredients, including vitamins, minerals, pharmaceuticals, and/or other active ingredients or precursors that are metabolized to active ingredients. The liposome can protect these compounds through the stomach into the intestines, where much higher concentrations are hence made available to the body. Liposomes can protect one or more certain active ingredients (e.g., active pharmaceutical ingredients) from external degradation, and similarity of the liposomes to biological membranes provides a way to deliver active ingredients into cells or subcellular compartments. What is more, various physicochemical properties of liposomes, including size, charge, and surface functional ligands, can be altered, resulting in functionalities favoring specific delivery tasks. Accordingly, such discrete phase particles provide an adaptable delivery platform for various active ingredients. Different antioxidant activities can provide synergistic effects in the management of various conditions. For example, the discrete phase particles including one or more compounds derived from olive oil can enhance the passage of an active ingredient into the body, including enhanced transdermal uptake as well as enhanced oral uptake. In certain embodiments, monounsaturated fats (e.g., oleic acid) present in the olive oil can improve the uptake and absorption of hydrophobic molecules or ingredients.

Certain benefits can be attributed to phenolic compounds of the olive oil, which can be due to the presence of secoiridoids; e.g., iridoids which are a type of monoterpenoid. Another health benefit stems from antioxidant activity and the protection that phenolic compounds exert on blood lipid oxidation. Further benefits and advantages can be attributed to anti-inflammatory activity, anti-carcinogenic potential, modulation of gene expression towards a protective mode for proteins participating in the cellular mechanisms involved in oxidative stress resistance, inflammation, or lipid metabolism. Such can also be attributed to other components of the olive oil, including hydroxytyrosol, tyrosol, oleuropein and aglycons, elenolic acid, as well as phenolic extracts rich in such compounds.

The various discrete phase particles provided herein can further include one or more of the following aspects. In certain embodiments, one or more saponins can be included, where saponins (a subclass of terpenoids) are amphipathic molecules and can act as surfactants. Saponins can interact with other components in layers and bilayers of the present discrete phase particles, including components such as cholesterol and phospholipids. Saponins include triterpene glycosides, including sugars attached to another organic molecule, usually a steroid or triterpene, a steroid building block. Particular examples include glycyrrhizin, quillaia, and squalene. Saponins can be sourced from various plant sources, including yucca. Squalene can be sourced from the olive oil described herein.

The various discrete phase particles provided herein can be multi-phasic or “multi-philic” and can include loading of multiple phase lipids. Phosphatidylcholine and polyethylene glycol containing formations especially allow for an interfacial region that can be hydrophilic, potentially allowing for the dual-layer encapsulation/loading of both hydrophilic and hydrophobic compounds of interest; e.g., active ingredients such as pharmaceutical ingredients. Cations (e.g., Mg²⁺) can increase the spontaneous formation of the interfacial region, stabilize a newly formed or preexisting interfacial region, and/or can allow for other interfacial interactions in colloidal chemistry when aqueous media are introduced.

The present technology further provides surprising and unexpected benefits attributable to utilization of cold process technology in preparation of olive oil. In order to preserve unstable and readily oxidizable fat content during sonication or nanotizing, a cold process can be utilized with chillers and re-circulation pumps. This can also reduce the relative phase inversion temperature, where such layer inversion is undesired, and can further assist in modulating structures in post-refinement and loading of lipid structures (e.g., various discrete phase particles provided herein) intended for delivery of one or more active ingredients of interest, including pharmacologic delivery.

The composition can be configured as an oral unit dosage form with an enteric coating in order to release the active ingredient after the stomach, for example, in the upper tract of the intestine. The enteric-coated composition can provide a sustained release dosage form. Unit dosage form examples include geltabs or capsules. Enteric coatings can function by presenting a surface that is stable at the acidic pH found in the stomach, but which can break down at a higher pH (e.g., more alkaline pH). For example, the enteric coating will not dissolve in the gastric acids of the stomach (e.g., pH ˜3), but will dissolve in the alkaline (e.g., pH 7-9) environment present in the small intestine. By preventing dissolution in the stomach, the enteric coating can also protect gastric mucosa from any irritating effects of one or more components of the composition itself. When the composition reaches the neutral or alkaline environment of the intestine, the coating can dissolve and components therein can be available for absorption into the bloodstream.

There are various ways to make enteric compositions. Enteric coatings can include one or more fatty acids, waxes, shellac, polymers, and/or plant fibers, as known in the art. Certain materials that can be used to formulate enteric coatings or enteric capsules include one or more of methyl acrylate-methacrylic acid copolymers, cellulose acetate phthalate, cellulose acetate succinate, hydroxypropyl methyl cellulose phthalate, hydroxypropyl methyl cellulose acetate succinate (hypromellose acetate succinate), polyvinyl acetate phthalate, methyl methacrylate-methacrylic acid copolymers, shellac, cellulose acetate trimellitate, sodium alginate, and zein. For example, an enteric coating aqueous solution (ethylcellulose, medium chain triglycerides [coconut], oleic acid, sodium alginate, stearic acid) can be used to form coated softgels.

There are various ways to make sustained release compositions. Such sustained release compositions can include dosage forms configured to release or liberate the active ingredient at a predetermined rate in order to maintain a constant concentration for a specific period of time with minimum side effects. This can be achieved through a variety of compositions, including the various discrete phase particles provided herein, along with and drug-polymer conjugates (e.g., hydrogels). Sustained or modified-release dosages can permit the active ingredient to dissolve over time in order to be released slower and steadier into the bloodstream. A further advantage is that the sustained release composition can be administered at less frequent intervals than an immediate-release composition of the same active ingredient. The sustained release nature of the composition can be particularly advantageous for oral dose compositions. Timed release has several distinct variants such as sustained release where prolonged release is intended, pulse release, delayed release (e.g., to target different regions of the gastrointestinal tract) etc. Sustained release not only prolongs action of the active ingredient, but can maintain active ingredient levels within a desired therapeutic window to avoid undesired or adverse peaks in active ingredient concentration following ingestion or injection, to thereby maximize therapeutic efficiency.

### EXAMPLES

Example embodiments of the present technology are provided below, including examples made with reference to the several figures enclosed herewith.

Various compositions of discrete phase particles that include at least one compound derived from Olea europaea can be prepared in various ways. The following preparation examples are provided solely as guidance, where one skilled in the art can adapt these methods as desired to include the various aspects described in the present disclosure.

Preparation of an embodiment of a composition, which can be formulated as an oral tincture, can be accomplished in three stages, denoted parts A, B, & C.

Part A: Connect a flowcell/sonicator to a 150 L planetary mixer via a re-circulator and set flow rate to 2-3 L/min. Connect 150 L planetary mixer jacketed liner or similar reactor to chiller/recirculation pump. Set recirculation pump rate to 10-15 L/min. Set chiller temperature to −3° C. Allow for both chiller/recirculation pump and flowcell/sonicator to operate at nominal capacity for the duration of sonication/nanotization.

In a lined drum, weigh out selected amount of carrier oils (e.g., olive oil, prim rose etc.) using a floor scale. Account for loss from adhesion to liner, feed lines, valves, etc. during transfer. Transfer olive oil and any additional oils into the 150 L planetary mixer using the transfer pump and new hoses. In separate appropriate containers, weigh out compound of interest (e.g., phenolic, isoprenic, and any other lipids to be encapsulated). In a separate appropriate container, weigh out phosphatidylcholine. Pulverize until particle weight is about <0.1 g.

For an active ingredient tincture, incorporate one or more desired active ingredients, such as a hydrophobic active pharmaceutical ingredient (API) into carrier oil along with phosphatidylcholine, prior to the incorporation of other actives. Shear until completely homogenized at high speed under low vacuum (e.g., about 0.02 Kpa) for approximately 90 minutes. It can be preferrable not exceed about 38° C. (˜100° F.) core temperature while shearing. Incorporate active ingredients and phosphatidylcholine into carrier oil. Shear until completely homogenized at high speed under low vacuum (e.g., about 0.02 Kpa) for approximately 90 minutes. Do not exceed 38° C. core temperature while shearing (e.g., 27° C. @ 3000 rpm). Allow to cool to ambient temperature.

Part B: This part includes rearranging fats, removing dissolved gases, water, and nanotizing micelles. In a separate container, incorporate volatiles (essential oils, terpenes, terpenoids and, sesquiterpenes). Mix at medium speed (e.g., 1700-2300 RPM) with propeller for 90 minutes using overhead bench mixer; using caution not to incorporate air into solution.

Part C: This part includes the addition of a heat sensitive active ingredient. After allowing Part A to cool to ambient temperature, incorporate volatiles from Part B into Part A. Mix at medium speed (e.g., 1700-2300 RPM) for 90 minutes using 150 L planetary mixer. Shear until completely homogenized. Do not apply vacuum while homogenizing volatiles. Filter Part C using a plate filter or supplied apparatus while transferring into final lined drum. Quarantine sample of filtered solution for testing. Apply tamper evident seal and protect from light until final test results arrive. Store in lined drums between −8° C. and −4° C. Make any adjustments needed to assay upon test results. Incorporate more carrier oil as needed.

As another example, a method for saponification of loaded phospholipids and oil is provided as follows. In its own container, measure surfactant (e.g., saponin) using a balance. In a main mixing container, weigh out deionized water. Using an overhead mixer and rotor stator, begin mixing (e.g., 1,500 rpm) and incorporate the surfactant until a homogeneous solution is formed. In a separate container, weigh out buffers to adjust pH for preservation. Incorporate buffers into main mixing container and mix until homogenous (e.g., 1,500 rpm). Add the product of Part C from above (the loaded lipid complex) into the water and surfactant and use rotor stator to induce high shear (e.g., 3,000 rpm). Mix until a polished and opalescent emulsion is formed. Keep core temperature less than 27° C. (using chiller) with mixing (e.g., 3,000 rpm). Remove rotor stator and insert sonication probe into emulsion and main mixing container in cold water bath. Sonicate at 80% power until all nucleation and air bubbles stop forming and all foam is resolved. Triturate while sonicating to assist in the removal of air. Keep core temperature less than 27° C. Remove sonication probe and allow to return to ambient temperature. Filter and apportion product as desired.

Certain advantages accrue based on the preceding methodology. By removing dissolved gases through sonication, shelf life is increased, where less oxidation of lipids occurs. A higher bioavailability of the active ingredient is achieved by forming lipid-based micelles in a lipid carrier or aqueous carrier. An increased efficacy/duration of the active ingredient is achieved by forming lipid-based micelles in a lipid carrier or aqueous carrier. The aqueous carrier molecules have a greater zeta potential and are pre-emulsified, enhancing absorption.

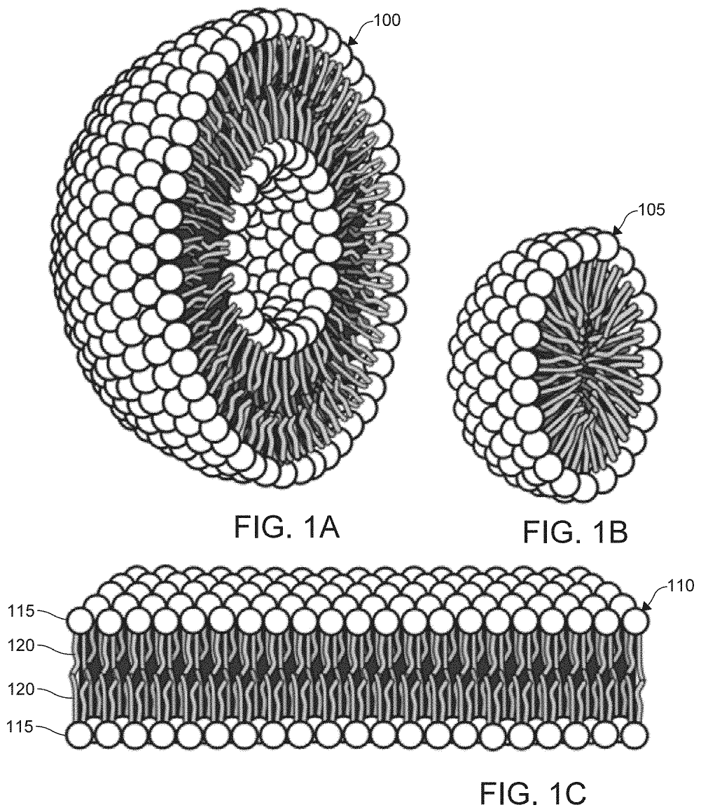

With reference now to FIGS. 1A, 1B, and 1C, schematic perspective cross-sections of the discrete phase particle are shown as a liposome 100 (FIG. 1A), a micelle 105 (FIG. 1B), and a bilayer sheet 110 (FIG. 1C), where phospholipids are depicted with hydrophilic heads 115 and hydrophobic tails 120.

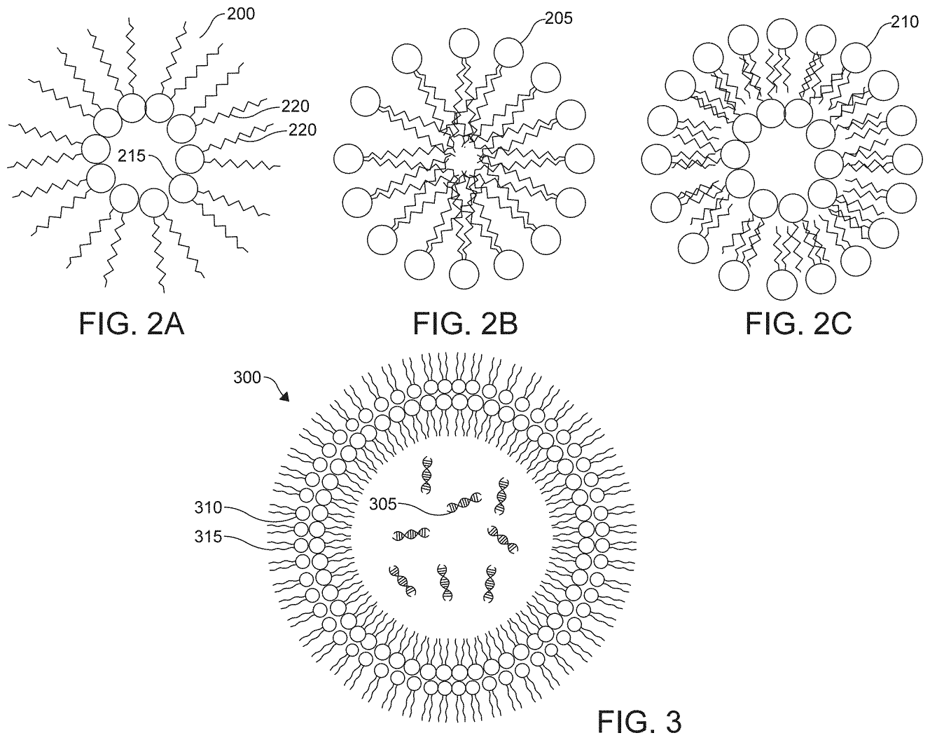

Turning now to FIGS. 2A, 2B, and 2C, schematic cross-sections of the discrete phase particle are shown as a reverse micelle 200 in aprotic continuous phase (FIG. 2A), a normal micelle 205 in an aqueous continuous phase (FIG. 2B), and a liposome 210 in an aqueous phase (FIG. 2C), where phospholipids are depicted with hydrophilic heads 215 and hydrophobic tails 220.

Turning now to FIG. 3, a schematic depiction of a reverse liposome 300 is shown having a reverse phospholipid bilayer with an active ingredient 305 enclosed by the reverse phospholipid bilayer, where phospholipids are depicted with hydrophilic heads 310 and hydrophobic tails 315.

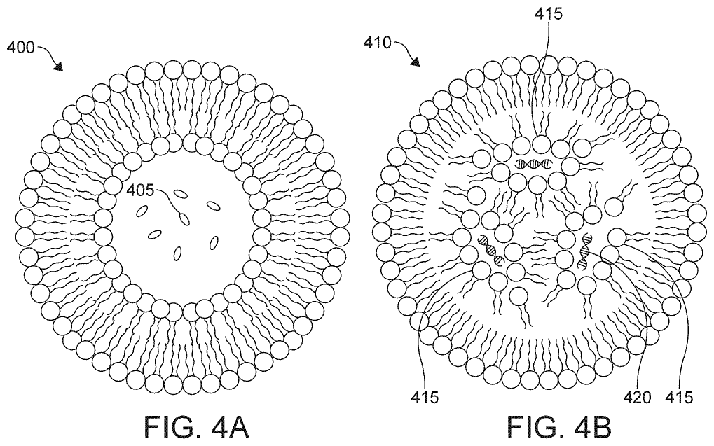

Turning now to FIGS. 4A and 4B, schematic cross-sections are shown of a liposome 400 including a bilayer enclosing an active ingredient 405 (FIG. 4A) and multilamellar discrete phase particle 410 including multiple enclosures 415 of an active ingredient 420 (FIG. 4B).

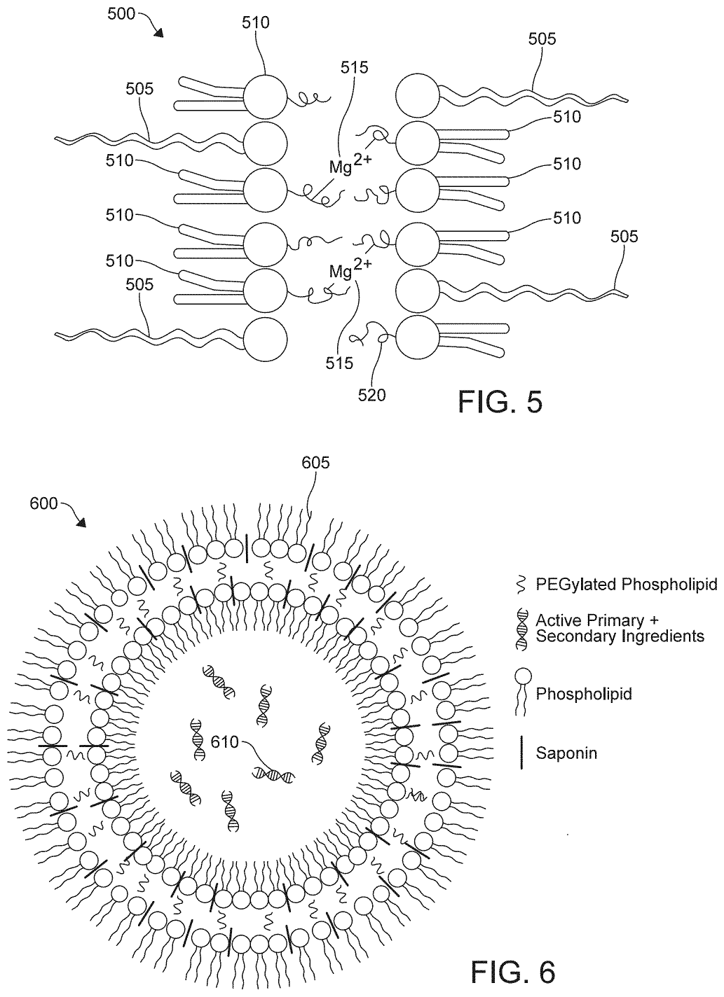

Turning now to FIG. 5, a schematic of a portion of a reverse bilayer 500 is shown, where saponin molecules 505 (represented by a single tail) are included with phospholipids 510 (represented by double tails) in the reverse bilayer 500, cations 515 (e.g., magnesium ions) provide bridging interactions between the polar or hydrophilic head portions of the reverse bilayer 500, and PEGylated phospholipids 520 are included in the reverse bilayer 500.

Turning now to FIG. 6, a schematic is shown of a reverse liposome 600 having a reverse phospholipid bilayer 605 with an active ingredient 610 enclosed by the reverse bilayer 605, where PEGylated phospholipids are present in the reverse bilayer 605 and saponin molecules are associated with the reverse bilayer.

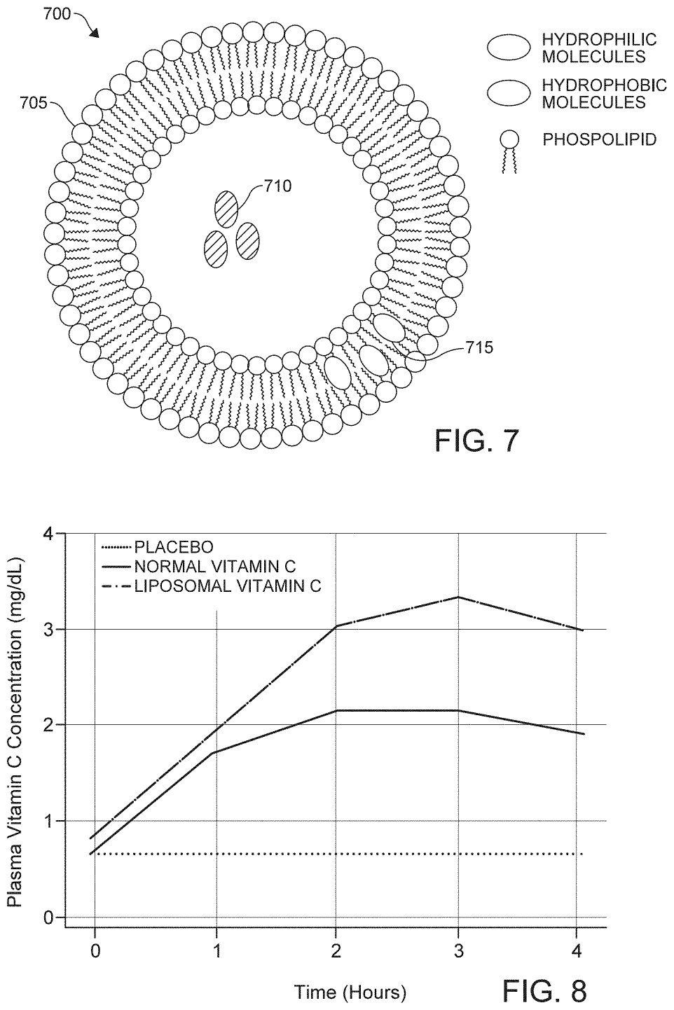

Turning now to FIG. 7, a schematic is shown of a liposome 700 having a phospholipid bilayer 705 with hydrophilic molecules 710 enclosed by the bilayer 705 and hydrophobic molecules 715 associated with the lipid portion of the bilayer 705.

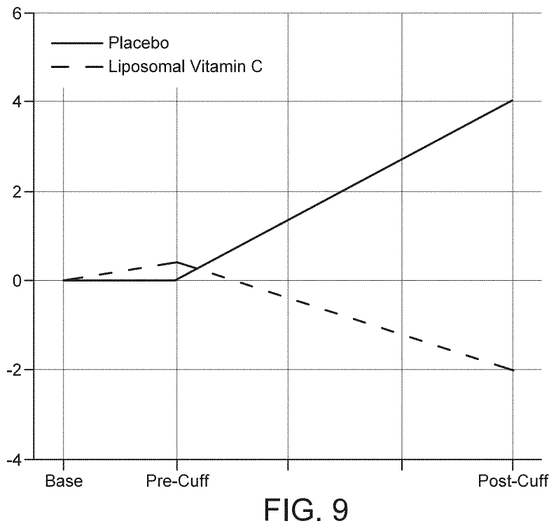

Turning now to FIGS. 8 and 9, graphical depictions of the uptake rate of vitamin C are shown. FIG. 8 shows data relating to a placebo of liposomes without vitamin C (dotted curve), liposomes containing vitamin C (dash-dot-dash curve), and normal vitamin C not contained by a discrete phase particle (solid curve). Certain advantages associated with liposomal delivery of vitamin C are illustrated thereby. While high doses of regular vitamin C powder or capsules will be rejected at high rates from the body, liposomal vitamin C can improve uptake. FIG. 8 shows that, compared with “normal” vitamin C ingestion (single dose of 4 g), ingestion of liposomal vitamin C (also 4 g) leads to appreciably greater circulating vitamin C concentrations. Hence, liposomes facilitate vitamin C delivery to the blood. With respect to vitamin C efficacy, FIG. 9 shows that the vitamin C concentration provides increased protection from oxidative stress, where a placebo (solid curve) and liposomal vitamin C (dotted curve) are compared. In this regard, an ischemia reperfusion model can be used. Ischemia describes any tissue that has been deprived of blood and oxygen. Reperfusion occurs when the blood and oxygen supply is returned. However, reperfusion can cause massive oxidative stress on the cardiovascular system, as the oxygen interacts with metabolites produced by the oxygen-deprived tissues. Per the liposomal vitamin C data (dotted curve) in FIG. 9, it is shown that oxidative stress can be reduced even during the increased oxidative stress of reperfusion.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A composition comprising:
a continuous medium forming a first phase; and
a discrete phase particle containing a second phase, the discrete phase particle including a compound derived from Olea europaea.

2. The composition of claim 1, wherein the first phase is an aqueous phase, the second phase is a nonaqueous phase, and the discrete phase particle is in the form of a micelle.

3. The composition of claim 1, wherein the first phase is a nonaqueous phase, the second phase is an aqueous phase, and the discrete phase particle is in the form of a reverse micelle.

4. The composition of claim 1, wherein the first phase is an aqueous phase, the second phase is a nonaqueous phase, the discrete phase particle is in the form of a liposome, and the liposome includes a third phase that is an aqueous phase.

5. The composition of claim 1, wherein the first phase is a nonaqueous phase, the second phase is an aqueous phase, the discrete phase particle is in the form of a reverse liposome, and the reverse liposome includes a third phase that is a nonaqueous phase.

6. The composition of claim 1, wherein the compound derived from Olea europaea includes a member selected from a group consisting of a phospholipid, a phenolic compound, a fatty acid, a terpenoid/isoprenoid compound, and combinations thereof.

1. position of claim 1, wherein the discrete phase particle further comprises an amphiphilic compound not derived from Olea europaea.

8. The composition of claim 1, wherein the discrete phase particle includes a PEGylated phospholipid.

9. The composition of claim 1, wherein the discrete phase particle further comprises saponin.

10. The composition of claim 1, wherein the discrete phase particle includes an active ingredient.

11. The composition of claim 10, wherein the active ingredient is present in the second phase.

12. The composition of claim 1, wherein the compound derived from Olea europaea includes olive oil.

13. The composition of claim 12, wherein the olive oil includes ice-pressed olive oil.

14. The composition of claim 13, wherein the ice-pressed olive oil is derived from Greek olives.

15. The composition of claim 14, wherein the ice-pressed olive oil derived from Greek olives includes a total amount of polyphenols (index D3) greater than about 500 mg/kg.

16. The composition of claim 14, wherein the ice-pressed olive oil derived from Greek olives includes a total amount of polyphenols (index D3) from about 500-2,000 mg/kg.

17. The composition of claim 14, wherein the ice-pressed olive oil derived from Greek olives comprises:
oleocanthal from about 130 mg/kg to about 245 mg/kg;
oleacein from about 90 mg/kg to about 165 mg/kg;
oleocanthal +oleacein (index D1) from about 220 mg/kg to about 410 mg/kg; and
total polyphenols analyzed (index D3) from about 760 mg/kg to about 1420 mg/kg.

18. The composition of claim 1, wherein the composition is encapsulated.

19. The composition of claim 1, wherein the composition is encapsulated by an enteric coating.

20. The composition of claim 1, wherein:
the compound derived from Olea europaea includes oleocanthal from about 130 mg/kg to about 245 mg/kg, oleacein from about 90 mg/kg to about 165 mg/kg, oleocanthal+oleacein (index D1) from about 220 mg/kg to about 410 mg/kg; and total polyphenols analyzed (index D3) from about 760 mg/kg to about 1420 mg/kg;
the discrete phase particle includes an active ingredient; and
the composition is encapsulated.

21. A method of administering an active ingredient to a subject, comprising:
providing a composition including:
a continuous medium forming a first phase;
a discrete phase particle containing a second phase, the discrete phase particle including a compound derived from Olea europaea; and
wherein the compound derived from Olea europaea includes oleocanthal from about 130 mg/kg to about 245 mg/kg, oleacein from about 90 mg/kg to about 165 mg/kg, oleocanthal+oleacein (index D1) from about 220 mg/kg to about 410 mg/kg; and total polyphenols analyzed (index D3) from about 760 mg/kg to about 1420 mg/kg; the discrete phase particle includes an active ingredient; and
administering the composition to the subject, thereby administering the active ingredient to the subject.

22. The method of claim 21, wherein the administering is topical.

23. The method of claim 21, wherein the administering is oral and the composition is encapsulated with an enteric coating.
